(19) United States
(12) Patent Application Publication
(10) Pub. No.: US 2021/0186399 A1
(43) Pub. Date: Jun. 24, 2021

(54) URINARY TRACT INFECTION DETERMINATION

(71) Applicant: Medtronic, Inc., Minneapolis, MN (US)

(72) Inventors: Bruce D. Gunderson, Plymouth, MN (US); Brian B. Lee, Golden Valley, MN (US); Andrew Radtke, Minneapolis, MN (US)

(21) Appl. No.: 16/720,487

(22) Filed: Dec. 19, 2019

(51) Int. Cl. A61B 5/20; A61B 5/11; A61B 5/0205; A61B 5/00; A61B 5/01

(52) U.S. Cl. CPC A61B 5/201; A61B 5/1115; A61B 5/204; A61B 5/0205; A61B 5/024; A61B 5/4809; A61B 5/01; A61B 2562/0219; A61B 5/4824

(57) ABSTRACT

Systems and methods for are disclosed for determining whether to output an indication of a urinary tract infection (UTI) in a patient, based on sensor data indicative of one or more common symptoms of UTIs, including, but not limited to, nocturia, fatigue or tremors; fever or chills; agitation or restlessness; lower back pain; painful urination; and presyncope or syncope.

### TECHNICAL FIELD

The invention relates to medical device systems and, more particularly, medical device systems for monitoring a condition of a patient.

### BACKGROUND

Many people suffer from urinary tract infections (UTIs). Over 10% of women older than 65 years of age reported having a UTI within the past 12 months. This number increases to almost 30% in women over the age of 85 years. When treated promptly and properly, lower UTIs rarely lead to complications. Left untreated, however, a UTI can have serious consequences. Complications of a UTI may include recurrent infections, especially in women who experience two or more UTIs in a six-month period or four or more UTIs within a year; permanent kidney damage from an acute or chronic kidney infection (pyelonephritis) due to an untreated UTI; increased risk in pregnant women of delivering low birth weight or premature infants; urethral narrowing (stricture) in men from recurrent urethritis, previously seen with gonococcal urethritis; and/or sepsis, a potentially life-threatening complication of an infection, especially if the infection works its way up the urinary tract to the kidneys. One example symptom of a UTI is nocturia, which is characterized by the need to urinate while a patient is sleeping, thereby possibly interrupting a sleep state of the patient.

### SUMMARY

In general, the invention is directed toward techniques for determining the onset or presence of a UTI in a patient based at least in part on data collected by one or more sensors. More specifically, a medical device system may use collected sensor data that is indicative of one or more symptoms associated with a UTI, and determine whether to provide an indication of UTI for the patient based on the sensor data. Some example UTI symptoms indicated by the sensor data may include an increased frequency of urination, particularly during nighttime, feelings of exhaustion or shakiness, fever or chills, agitation and restlessness, lower back pain, or a painful or burning sensation during urination, among several others.

In one example, a method includes detecting out-of-bed events of a patient based on sensor data from at least one sensor device; determining a frequency of the detected out-of-bed events for the patient; and determining, based on the frequency of out-of-bed events for the patient, to provide a urinary tract infection (UTI) indication for the patient.

In one example, a system includes at least one sensor device configured to collect sensor data; and processing circuitry configured to detect out-of-bed events of a patient based on the sensor data; determine a frequency of the detected out-of-bed events for the patient; and determine, based on the frequency of out-of-bed events for the patient, to provide a urinary tract infection (UTI) indication for the patient.

The details of one or more examples are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a system configured to monitor for the presence of a UTI in a patient, in accordance with some techniques of this disclosure.

FIG. 2 is a block diagram illustrating an example computing device configured to monitor for the presence of a UTI in a patient, in accordance with some techniques of this disclosure.

FIG. 3 is a flow diagram illustrating an example method of determining whether to provide an indication that a UTI is present in a patient, in accordance with techniques of this disclosure.

FIG. 4 is a flow diagram illustrating an example method of determining a number of interruptions in a sleep state of patient, according to techniques of this disclosure.

### DETAILED DESCRIPTION

UTIs are a condition that affects the quality of life and health of many people. One symptom of UTIs is an increased frequency of urination, often due in part to a decreased volume of urine voided during each urination event. The urge or need to void during a sleep event may be referred to as nocturia, or “sleep-event voiding.” Sleep-event voiding may disrupt a patient's sleep because of a reoccurring need or urge to void during a sleep state, which in turn may affect the patient's quality of life. The failure to get a full night of rest may adversely impact the patient's performance during the day, such as by causing fatigue or inattentiveness. Other UTI symptoms include a fever or chills, agitation, lower back pain, or a painful or burning sensation during urination, among several others.

Techniques are described herein for collecting sensor data indicative of one or more symptoms of UTI, such as nocturia, fatigue, fever, and painful urination, among others, and determining, based on the symptoms, a probability that a patient has a UTI, or otherwise determining whether to provide an indication that the patient has UTI. The techniques described may also be useful for evaluating the extent of the patient's nocturia by creating a log of the number of times the patient's sleep state is interrupted during a sleep event. The sleep event is generally measured between the commencement of a sleep state, such as when the patient begins attempting to sleep (e.g., a sleep initiation state), until the end of the sleep state, such as when the patient wakes up in the morning (although it need not necessarily be morning). As described in further detail below, the end of the sleep state is distinguishable from interruptions in the sleep state, after which the patient returns to the sleep state.

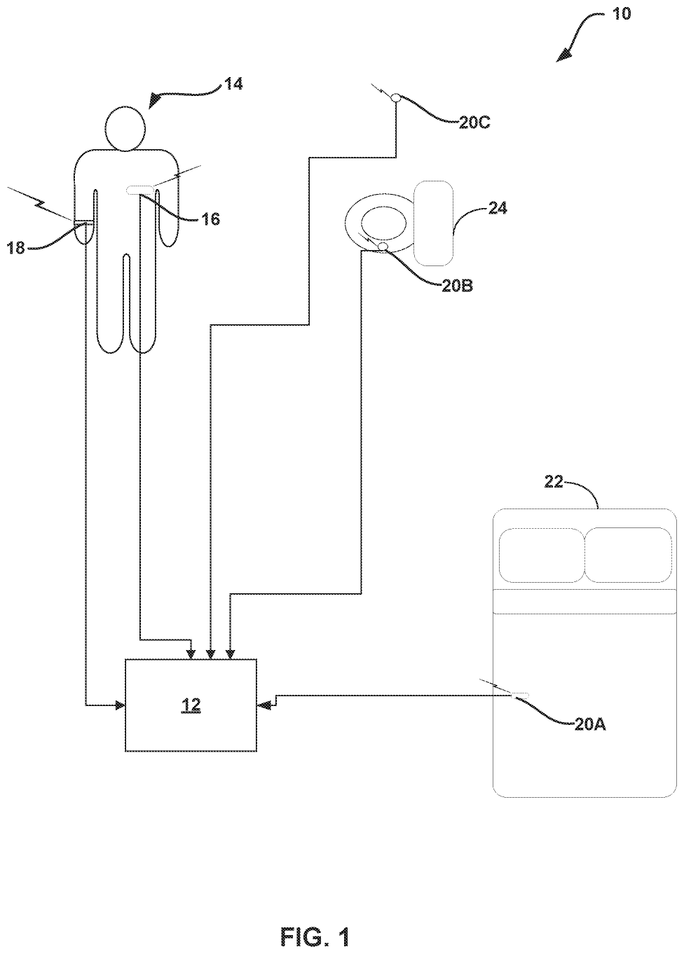

FIG. 1 is a schematic diagram illustrating an example UTI detection system 10, including a computing device 12 configured to determine whether a patient 14 likely has a UTI, based on data collected by or using one or more sensor devices. Sensor devices may include a wide variety of different types of sensors, configured to collect various data indicative of one or more physical parameters or behaviors of patient 14. For example, sensor devices may include an implantable sensor device 16 within the body of patient 14, a wearable sensor device 18 worn by patient 14, or any number of external sensor devices s 20A-20C (collectively, “external sensor devices 20”) disposed around an environment of patient 14.

An implantable sensor device 16 may take the form of an implantable medical device (IMD), such as a cardiac monitor having electrodes configured to collect data, such as a subcutaneous electrocardiogram (ECG) signal and/or a cardiac electrogram (EGM) signal indicative of electrical activity of a heart of patient 14, including data regarding heart rate, heart rate variability, and arrhythmic episodes. IMD 16 may also be configured with one or more sensors to collect other physiological data, such as one or more accelerometers configured to detect movement, steps, and posture/orientation, one or more temperature sensors, electrodes to sense respiration or mechanical activity of the heart, or one or more optical sensors (photoplethysmography (PPG) sensors) to sense oxygen saturation or mechanical activity of the heart.

One example of a cardiac monitor is the Reveal LINQ™ Insertable Cardiac Monitoring System, available from Medtronic plc. The Reveal LINQ™ Insertable Cardiac Monitoring System is an example of a cardiac monitor that includes electrode configured to sense a subcutaneous ECG, as well as other sensors. Other examples of implantable sensor device 16 include devices configured as pacemakers, cardioverters, and/or defibrillators, which may include one or more electrodes positioned on, within, or near the heart, e.g., via one or more leads, to sense a cardiac EGM. Such devices may include additional sensors as described herein.

System 10 may also include wearable sensor device 18, depicted in FIG. 1 as a wrist-wearable activity monitor, including one or more accelerometers (inertial measurement unit, or IMU), pedometers, PPG sensors, and/or other sensors, e.g., the same as or different than IMD 16. System 10 may also include a plurality of external sensor devices 20, such as sensor device 20A disposed within a bed 22, configured to indicate the presence of patient 14 within the bed based on, e.g., pressure, temperature, motion, sound, and/or image analysis. Another example external sensor device 20 of system 10 may include a pressure sensor device 20B disposed within a seat of a toilet 24, configured to indicate the presence of patient 14 on the toilet during a bladder-voiding event. Another example external sensor device 20 of system 10 may include one or more motion sensor devices 20C disposed anywhere between a bedroom and a bathroom of patient 14, configured to detect a motion of patient 14 towards or away from the bathroom for a bladder-voiding event. In other examples, external sensor device 20C may include a microphone configured to collect audio indicative of the presence of patient 14 within the bathroom, or in particular, of a bladder-voiding event. The example depicted in FIG. 1 is not intended to be limiting. Other example systems in accordance with this disclosure may include more, fewer, or different sensor devices or other components than those depicted in FIG. 1. For example, other types of sensor devices not shown in FIG. 1 may include a magnetometer (e.g., a compass) worn by patient 14, configured to indicate a direction of motion of patient 14 either toward or away from the bathroom for a bladder-voiding event.

UTI detection system 10 includes computing device 12 configured to receive sensor data from any or all of sensor devices 16, 18, 20. Computing device 12 may include any device having a memory and processing circuitry configured to receive sensor data and process the data according to the techniques of this disclosure. For example, computing device 12 may include a personal computing device, such as a smartphone, tablet, or laptop. Computing device 12 may include a remote server, such as managed by a medical practice or practitioner, or a manufacturer of one of sensor devices 16, 18, 20, such that a physician for patient 14 may access and view the data so as to inform treatment of patient 14 as needed. In some examples, computing device 12 may be integrated within one or more of sensor devices 16, 18, 20. In some examples, some or all of the functionality described herein as being performed by computing device (e.g., by processing circuitry of the computing device) may be performed by one or more of sensor devices 16, 18, 20 (e.g., by processing circuitry of the one or more sensor devices). For example, one or more of sensor devices 16, 18, 20 may independently or cooperatively identify when patient 14 is sleeping, identify interruptions of a sleep state, determine whether the interruptions qualify as out-of-bed events, and report numbers and/or rates of out-of-bed events to computing device 12.

In some examples in accordance with this disclosure, computing device 12 is configured to receive sensor data from sensor devices 16, 18, 20, and process the data in order to identify (e.g., determine an indication of), one or more symptoms of a UTI. The sensor data may include digitized versions of real-time sensor signals, and/or data determined by the sensor devices from the sensor signals. In some examples, computing device 12 is configured to identify any or all of seven common UTI symptoms: (1) an increased frequency of urination events and/or a reduced volume of urination per event; (2) fatigue and/or tremors; (3) fever and/or chills; (4) agitation and/or restlessness; (5) lower back pain; (6) painful urination (e.g., a “burning” sensation); and/or (7) presyncope and/or syncope (e.g., sudden loss of consciousness and/or falls). In some examples, system 10 is configured to determine that the probability of a UTI in patient 14 merits providing an indication of UTI, e.g., to a physician or other caregiver, by identifying at least an increased frequency of urination events by monitoring the patient's out-of-bed events over a period of time.

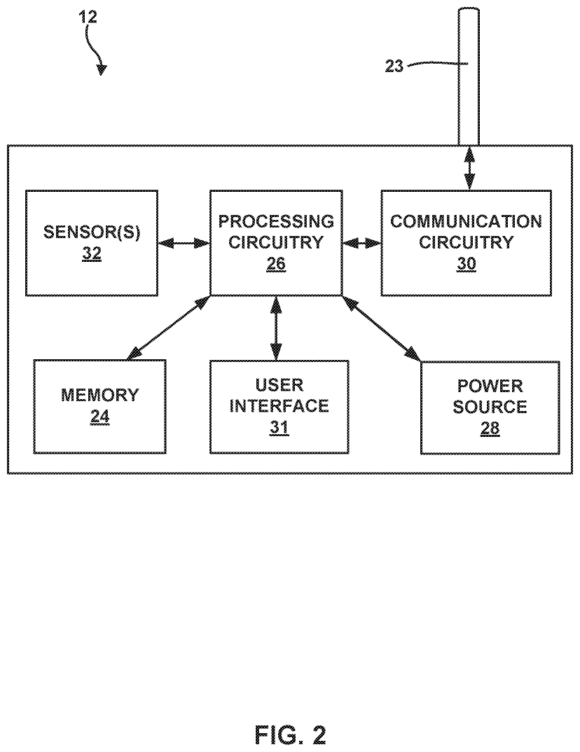

FIG. 2 is a block diagram illustrating an example computing device 12 configured to determine the presence of a UTI in a patient, in accordance with techniques of this disclosure. Computing device 12 includes a communication element 23, memory 24, processing circuitry 26, power source 28, communication circuitry 30, a user interface 31, and in some examples, one or more integrated sensor(s) 32. Although communication element 23 is depicted in FIG. 2 as an antenna, communication element 23 may include any physical component configured to facilitate communication with, e.g., to receive sensor data from, any of sensor devices 16, 18, 20 (FIG. 1). Communication element 23 may facilitate wireless and/or wired connections to sensor devices.

Memory 24 of computing device 12 may include any volatile or non-volatile media, such as any one or more of a random access memory (RAM), read-only memory (ROM), nonvolatile RAM (NVRAM), electronically-erasable programmable ROM (EEPROM), flash memory, and the like. Memory 24 is configured to store sensor data as well as instructions that, when executed by processing circuitry 26, cause processing circuitry 26 to process the stored sensor data to identify sensor data indicative of one or more symptoms of a UTI, and based on the analysis of the sensor data, determine whether a UTI is likely present in a patient, e.g., determine a probability that a patient has a UTI. Memory 24 may also store data generated by sensor devices 16, 18, 20 (FIG. 1) and/or processing circuitry 26.

Processing circuitry 26 may include any one or more of a microprocessor, a controller, a digital signal processor (DSP), an application specific integrated circuit (ASIC), a field programmable gate array (FPGA), discrete logic circuitry, or the like. Processing circuitry 26 is configured to execute one or more sets of instructions according to the techniques of this disclosure. For example, processing circuitry 26 may be configured to process sensor data indicative of one or more symptoms of a UTI, and based on the sensor data, determine to provide an indication that a patient has a UTI. For example, processing circuitry 26 may configured to determine the presence of a UTI in a patient by identifying at least an increased frequency of nighttime urination events (e.g., nocturia) by monitoring the patient's out-of-bed events over a period of time.

For example, processing circuitry 26 may be configured to receive, via communication element 23 and communication circuitry 30 from at least one sensor device 16, 18, 20 (FIG. 1), sensor data indicative of one or more out-of-bed events for a patient 14, and determine, based on the sensor data, a relative frequency of out-of-bed events for the patient 14. Processing circuitry 26 may further determine, based at least in part on the frequency of out-of-bed events, whether to provide an indication that the patient has a urinary tract infection (UTI).

For example, processing circuitry 26 may receive sensor data that relates to the activity of the patient 14 in order to determine a number of interruptions in a sleep state of patient 14. In particular, as will be described in greater detail below, processing circuitry 26, or processing circuitry of one of sensor devices 16, 18, 20, monitors an activity level of patient 14 in order to determine when patient 14 transitions from a sleep state to an awake state and back to the sleep state, which thereby indicates that the patient has awoken to void, and then returned to bed rather than ending the sleep state. An “awake” state does not imply any particular level of consciousness, but rather that physical activity was undertaken by the patient, e.g., to void. The transitions between the sleep state and awake state are used to determine whether the patient's sleep is interrupted because of nocturia. Typically, the relevant determination is whether the patient's sleep is interrupted because of a need or desire to get out of bed to void. Thus, the “awake state” refers to a state in which patient 14 is not only not asleep, but is active. The activity level is selected to reflect a patient activity level that occurs when patient 14 moves from, e.g., a bed to a bathroom to void or gets out of bed. While the interruption in the sleep state may be attributable to other reason, such as to answer a telephone call, the activity level is used in the present invention as a general indicator of interruptions in the sleep state, which generally represent interruptions due to nocturia, one common symptom of a UTI.

In some examples, processing circuitry 26 may be configured to receive sensor data from an activity-level monitor, such as IMD 16 (FIG. 1) or a wearable wrist sensor 18, indicating an activity level of patient 14. Processing circuitry 26 may analyze the received sensor data in order to identify an activity-level pattern indicative of an out-of-bed (OOB) event. For example, an OOB event may manifest within sensor data as (1) a relatively low activity level, while the patient is asleep; (2) a relatively high activity level while the patient travels from a bed to a toilet; (3) a relatively low activity level while the patient voids their bladder; (4) a relatively high activity level while the patient travels from the toilet back to the bed; and (5) a relatively low activity level while the patient resumes a sleep state. Processing circuitry 26 may analyze the received sensor data to determine one or more instances of this down-up-down-up-down activity-level pattern, and maintain a log in memory 24 indicating, for example, the number, time, and/or frequency (e.g., OOB events per day) of these identified OOB events. Processing circuitry 26 may analyze the frequency of OOB events over time (e.g., over a series of days) based on the log stored in memory, wherein a higher current or shorter-term average frequency of OOB events relative to the baseline, typical, or longer-term average frequency for patient 14 correlates to a higher probability of a UTI.

In some examples, alternatively or in addition to determining the down-up-down-up-down activity-level pattern described above, processing circuitry 26 may be configured to analyze additional aspects of the accelerometer data or other activity data in order to confirm that the activity pattern is likely due to a bladder-voiding event, rather than a different nighttime activity. For example, processing circuitry 26 may identify a number of steps during the first or second increased activity level, and determine whether the number of steps is indicative of (e.g., falls within a threshold window of) the patient walking between the bedroom and the bathroom. In another example, processing circuitry 26 may be configured to receive time duration data, e.g., from a clock or timer, collected during one or more of the detected activity levels, and determine whether the time duration is indicative of, e.g., falls within a threshold window of, the patient walking to the bathroom, voiding a bladder, or returning to the bedroom, respectively. In another example, processing circuitry 26 may be configured to determine, based on the received activity-level data, whether a cumulative activity level amount (e.g., the detected activity level integrated over the time during which the level is detected) is indicative of the patient walking to and/or from the bathroom. In some examples, processing circuitry 26 may identify an OOB event based on the activity data, e.g., level, steps, or duration, falling within threshold window that is determined statistically based on previous OOB events of the patient or of a population of patients.

Additionally or alternatively to receiving activity-level sensor data, processing circuitry 26 may be configured to receive sensor data from a position and/or orientation sensor, indicating, for example, a location and/or direction of motion of patient 14. For example, a position sensor may include any sensor device configured to indicate a relative location of patient 14. For example, the position sensor could include a mobile device, such as a smartphone, of patient 14, wherein the mobile device uses high-accuracy GPS and/or Wi-Fi signals to determine a room of patient 14 within a dwelling (e.g., a bedroom or a bathroom). A position sensor may also include an RFID tag configured to be scanned or read by an RFID reader device fixed in a known location, or other similar position indicator. An orientation sensor may include a magnetometer or compass, such as within a mobile device (e.g., smartphone), configured to indicate a direction of motion of patient 14 while patient 14 is in possession of the device. In some examples, any or all of these sensor devices may be integrated within a single IMD.

Processing circuitry 26 may analyze the received sensor data in order to identify a position/orientation pattern indicative of an OOB event. For example, an OOB event may manifest within position/orientation sensor data as any or all of (1) the presence of patient 14 within a bedroom; (2) a known direction of motion of patient 14 from the bedroom to a bathroom; (3) the presence of patient 14 within the bathroom; (4) a known direction of motion from the bathroom back to the bedroom; and (5) the presence of patient 14 back in the bedroom.

In some examples, processing circuitry 26 may be configured to identify an OOB event based on received sensor data indicating the presence of patient 14 within a bathroom. For example, processing circuitry 26 may identify an OOB event based on sensor data received from a pressure sensor 20B oriented underneath the seat of a toilet, indicating the presence of patient 14 seated on the toilet. In some examples, processing circuitry 26 may be configured to compare the pressure-sensor data with previous pressure data stored in memory 24, in order to verify that patient 14 is the individual currently seated on the toilet, rather than another member of the household.

Alternatively or additionally, processing circuitry 26 may identify or confirm an OOB event based on sensor data received from a motion detector 20C placed near or within the bathroom. Alternatively or additionally, processing circuitry 26 may identify or confirm an OOB event based on sensor data including audio data received from a microphone, such as worn by patient 14 or fixed near the toilet, indicating sounds indicative of a bladder-voiding event. In some examples, processing circuitry 26 may be configured to determine a volume (e.g., quantity) of urine based on, for example, the amplitude (e.g., volume) of the audio data and/or the duration of the sound.

Alternatively or additionally, processing circuitry 26 may be configured to identify an OOB event based on received sensor data indicating the presence and/or absence of patient 14 within a bedroom during a nighttime period. For example, processing circuitry 26 may receive sensor data from a pressure sensor 20A embedded within a bed, in order to determine when patient 14 is within the bed or, instead, is voiding their bladder. Because more than one individual may be present within a bed or a bathroom, processing circuitry 26 of any of the previous examples may be configured to identify a specific patient 14 based on short-range wireless communications (e.g., RFID, Bluetooth, etc.) with a device, such as a mobile device, wearable device, or implantable medical device, positioned on the person of patient 14.

Additionally or alternatively to determining nocturia by monitoring a frequency of OOB events for patient 14, processing circuitry 26 may be configured to determine whether to provide an indication of UTI based at least in part on received sensor data indicating a level of fatigue and/or tremors for patient 14. For example, due to frequent interruptions to their nighttime sleep state, patient 14 may experience an increased level of fatigue during the daytime. In some examples, high levels of fatigue may manifest observable symptoms such as tremors, for example, a shaking of the hands or other body parts of patient 14, which may be detectable via one or more accelerometers of wearable sensor device 18 for example.

In some examples, any the same types of activity-level sensors as described above may be configured to collect and transmit sensor data indicative of a daytime activity level for patient 14. Processing circuitry 26 may be configured to receive the activity-level sensor data and identify a relative decrease in daytime activity level, indicative of a respective increased level of fatigue. In some examples, processing circuitry 26 may be configured to identify, based on the received activity-level sensor data, one or more periods of time during which patient 14 is walking, and also determine from the sensor data a decreased average walking speed, indicative of respective increased level of fatigue.

In some examples in which a sensor device includes a body-position sensor configured to indicate a relative position of the body of patient 14 (e.g., sitting, standing, lying down), processing circuitry 26 may be configured to determine, based on received sensor data, an increase in the duration of patient 14 sitting and/or lying down relative to standing, indicative of an increased level of fatigue. For example, processing circuitry 26 may identify an increased sitting-time-to-standing-time ratio, or similarly, an increased total-lying-down-duration.

In some examples in which a sensor device includes an activity-level sensor or other sensor device configured to identify a sleep state (e.g., electroencephalogram (EEG) sensors, heart rate sensors, respiration sensors, etc.), processing circuitry 26 may be configured to determine an increased level of fatigue for patient 14 based on received sensor data indicating, for example, an earlier-than-average sleep time, a later-than-average wake-up time, and/or a longer-than-average total sleep duration (e.g., the difference between a final wake-up time and a first fall-asleep time).

As mentioned above, an increased level of fatigue may, in some examples, correspond to other observable symptoms in patient 14, such as tremors (e.g., shaking). For example, processing circuitry 26 may be configured to identify tremors, based on received sensor data from an activity-level sensor, indicating an increased baseline-activity level. For example, an increased baseline activity level may indicate that the patient's body is shaking while otherwise at rest, which may indicate an increased level of fatigue, which may indicate poor sleep quality due to nocturia, which may indicate an increased probability of UTI.

Additionally or alternatively to determining nocturia by monitoring a frequency of OOB events for patient 14, processing circuitry 26 may be configured to determine whether to provide an indication of UTI based at least in part on received sensor data indicating a fever and/or chills, another common symptom of a UTI. For example, a wearable or implanted sensor device may include a temperature sensor configured to detect an internal body temperature for patient 14. Processing circuitry 26 may be configured to determine an increased probability of a UTI based at least in part on received sensor data indicating an increased internal body temperature. In another example, processing circuitry 26 may be configured to receive sensor data from both a temperature sensor and an activity-level sensor, and more-accurately determine that patient 14 has a fever by identifying that patient 14 has an increased body temperature even while at rest (e.g., determining that an increased activity level is not the cause of the increased body temperature).

Additionally or alternatively to determining nocturia by monitoring a frequency of OOB events for patient 14, processing circuitry 26 may be configured to determine whether to provide an indication of a UTI based at least in part on received sensor data indicating an increased level of agitation or restlessness, due to pain or discomfort caused by the UTI. For example, the pain or discomfort of a UTI may prevent patient 14 from falling into a deep sleep, or in some cases, falling asleep at all. Accordingly, processing circuitry 26 may be configured to identify a UTI based at least in part on received activity-level sensor data indicating an increased number of above-threshold activity windows during a sleep duration. The above-threshold activity windows may indicate the patient tossing and turning during the night, unable to find enough comfort to fall asleep. Similarly, in examples in which a sensor device includes a body-position sensor, processing circuitry 26 may be configured to identify a UTI based on increased number of bodily turns of patient 14 during a sleep duration. For example, processing circuitry 26 may be configured to identify, based on received body-position sensor data, instances in which patient 14 has transitioned from a face-down orientation to a face-up orientation, and/or vice versa, and maintain a lot indicating the number and/or frequency of these turns over a nighttime period.

Additionally or alternatively to determining nocturia by monitoring a frequency of OOB events for patient 14, processing circuitry 26 may be configured to determine whether to provide an indication of UTI based at least in part on received sensor data indicating an acute lower back pain of patient 14. For example, processing circuitry 26 may be configured to identify one or more behaviors of patient 14 indicative of a constant or near-constant lower back pain, another common symptom of a UTI.

For example, processing circuitry 26 may be configured to receive sensor data from a body-position sensor as described above, and determine, from the sensor data, a change in a patient's average posture. For example, processing circuitry 26 may determine from body-position sensor data a duration of when patient 14 is seated and when they are standing throughout a given period of time, such as a day. Processing circuitry 26 may then determine that the average amount of time that patient 14 is seated rather than standing has changed, indicative of discomfort in a particular posture due to lower back pain from a UTI. For example, the position sensor data may indicate an increased frequency of change in body position, due to patient 14 frequently switching between a seated and a standing posture in response to lower back pain or other UTI-related discomfort.

Similar to a determination of increased levels of fatigue as described above, processing circuitry 26 may further be configured to determine that patient 14 is experiencing lower back pain by identifying, based on activity-level sensor data, a decreased average gait pace for patient 14. For example, since sudden movements may exacerbate lower back pain, processing circuitry 26 may be configured to identify, based on received activity-level sensor data, one or more periods of time during which patient 14 is walking, and also determine from the sensor data a decreased average walking speed, indicative of lower back pain due to a UTI.

In another example, in which a sensor device includes a body-position sensor, processing circuitry 26 may be configured to determine lower back pain for patient 14 based on received sensor data indicating, for example, a decreased variation of posture vectors about an upright reference. For example, for some individuals experiencing lower back pain from a UTI, body postures such as slouching may exacerbate the pain. Accordingly, patient 14 may exhibit an increased likelihood to sit, stand erect, or vary between the multiple body positions in order to partially relieve the pain or discomfort, as compared to a “normal” or previous body posture. In these examples, processing circuitry 26 may be configured to identify, based on received body-position sensor data, a number of different body postures of patient 14 as well as the duration spent in each respective posture, and identify, based on the postures and durations, that patient 14 is spending an increased amount of time in more-upright body postures.

In another example, processing circuitry 26 may be configured to determine whether patient 14 is experiencing lower back pain by determining, based on sensor data, that patient 14 has ingested a pain-relieving drug. For example, an implantable cardiac monitor may output sensor data indicative of an increased heart rate (HR) and/or a decreased heart-rate variability (HRV), two common effects of a pain-relieving drug on the body. Processing circuitry 26 may be configured to compare HR and HRV data with other sensor data, such as activity-level sensor data, in order to determine that patient 14 is experiencing an increased heart rate while physically at rest, such that an increased level of physical activity is not responsible for the increased heart rate, and instead, is attributable to the effects of a drug.

Additionally or alternatively to determining nocturia by monitoring a frequency of OOB events for patient 14, processing circuitry 26 may be configured to determine whether to provide an indication of a UTI based at least in part on received sensor data indicating painful bladder-voiding events (e.g., urination) for patient 14, another common symptom of a UTI. For example, some patients may experience an increased heart rate in reaction to acute pain during urination. Accordingly, processing circuitry 26 may be configured to receive sensor data from an implantable cardiac monitor indicating an increased heart rate during urinary events, as determined by other received sensor data, as described above with respect to out-of-bed events.

Additionally or alternatively to determining nocturia by monitoring a frequency of OOB events for patient 14, processing circuitry 26 may be configured to determine whether to provide an indication of a UTI based at least in part on received sensor data indicating effects of infection, such as increased temperature (described above), increased HR, increased respiration rate, and/or decreased HRV. In some examples, IMD 16 and/or another sensor device may be configured to determine respiration rate, e.g., using electrodes to detect changes in impedance or optical sensors to detect respiration via photoplethysmography.

Additionally or alternatively to determining nocturia by monitoring a frequency of OOB events for patient 14, processing circuitry 26 may be configured to determine whether to provide an indication of UTI based at least in part on received sensor data indicating presyncope and/or syncope, or in other words, a feeling of light-headedness, or even a total loss of consciousness and/or a respective fall due to loss of consciousness. As described in commonly-assigned U.S. Patent Application No. 62/854,086, entitled, “MEDICAL DEVICE FOR FALL DETECTION” to Michelle M. Galarneau, incorporated herein by reference in its entirety, processing circuitry 26 may determine, based at least in part on sensor data received from one or more accelerometers, an indication of one or more falls by patient 14. For example, one or more accelerometers and/or other body-position sensors may indicate a sudden deviation (an above-threshold acceleration away) from an “upright” body posture, indicative of a fall. Additionally or alternatively, since loss of consciousness and/or falls may be caused by a sudden drop in blood pressure, processing circuitry 26 may determine an indication of a fall based on sensor data received from a cardiac monitor indicating such a sudden drop. Processing circuitry 26 may be configured to provide an indication of a UTI based on a higher identified number and/or frequency of falls.

Processing circuitry 26 may be configured to analyze frequency of OOB events and/or additional sensor data indicative of additional UTI symptoms, and determine whether to provide an indication of UTI for patient 14 based on the analysis. In some examples, the analysis may include identifying whether values of OOB frequency or other UTI symptom data satisfy a threshold or other criterion consistent with UTI. In some examples, the threshold may be predetermined, e.g., user-programmable. In some examples, processing circuitry 26 may determine the threshold based on previous values of OOB frequency or other UTI symptom data (e.g., temperature, daytime activity, HR, HRV, etc.), determined by processing circuitry 26 for patient 14 and/or for a population of patients. For example, processing circuitry 26 may determine a threshold based on a mean, median, or other average of previous values of patient 14. In this manner, satisfaction of the threshold may indicate a deviation from a baseline or baseline trend of patient 14 that is indicative that patient 14 has developed a UTI. In some examples, the threshold(s) may be determined statistically, e.g., the threshold value may be associated with a target probability of UTI in a patient population.

In some examples, processing circuitry 26 may calculate the frequency of OOB events each night (e.g., OOB per hour) and store a baseline trend (e.g., over 14 days) in memory 24. Processing circuitry 26 may then use statistical process control (SPC) to determine a “normal” range of baseline trend values. A relatively high value as determined via SPC may indicate an acute OOB frequency which could potentially result from a UTI. For example, a UTI may trigger a relatively sudden onset of symptoms, resulting in an abrupt change in one or more sensor data values. This algorithm may provide a relatively high sensitivity when determining a potential UTI in patient 14. In some examples, processing circuitry 26 may incorporate additional variables to improve the specificity. For example, processing circuitry may use similar techniques to identify aa statistically significant change in the other variables (e.g., increased sleep restlessness, increased body temperature, increased HR, decreased HRV). In some examples, processing circuitry 26 may require the identification of a significant change in at least one additional variable in order to “confirm” a UTI determination. In some examples, in response to making a positive UTI determination, processing circuitry 26 may output a prompt for a user (e.g., patient 14 or a physician of patient 14) to collect and submit additional sensor data, such as a manual body temperature measurement or other UTI symptom indicator. Upon receiving additional data input, processing circuitry 26 may analyze the additional data to either “confirm” (e.g., determine an increased probability) or “reject” (e.g., determine a decreased probability) the initial UTI determination.

Processing circuitry 26 may be coupled to power source 28. Power source may include a battery and/or wired power connection. In examples in which computing device is integrated within a sensor device, such as implantable sensor device 16, power source 28 may take the form of a small, rechargeable or non-rechargeable battery, or an inductive power interface that transcutaneously receives inductively coupled energy. In the case of a rechargeable battery, power source 28 similarly may include an inductive power interface for transcutaneous transfer of recharge power.

Communication circuitry 30 is configured to exchange telemetry information with an external programmer, such as a clinician programmer and/or a patient programmer by wireless telemetry. In addition, in some examples communication circuitry 30 supports wireless communication with one or more wireless sensor devices that generate signals indicative of physiological parameters or motion of patient 14 and transmit the signals to computing device 12. Such communication may be via any wireless communication protocol, such as known medical device telemetry protocols, or the Bluetooth™ protocol.

In some examples, computing device 12 includes one or more sensor(s) 32. Sensor(s) 32 may include any one or more of the example sensors discussed herein with respect to sensor devices 16, 18, and/or 20 (FIG. 1). For example, one or more sensor(s) 32 may be configured to generate a signal indicative of a patient activity level, e.g., may include one or more accelerometers.

Sensor(s) 32 and/or sensors within sensor devices 16, 18, and 20 configured to detect patient activity may be any sensor device such as an accelerometer (e.g., one or more multiple axis accelerometers or one or more single axis accelerometers arranged along one or more axes), a bonded piezoelectric crystal, a mercury switch, or a gyro, or any other sensor device that transforms mechanical, chemical or electrical conditions into electrical signals representative of an activity level of patient 14. A multiple-axis accelerometer, also referred to as a multi-axis accelerometer, or multiple single axis accelerometers may be useful for generating a signal that may be used to determine both a patient activity level and a patient posture. The electrical signals from sensors may be amplified, filtered, and otherwise processed as appropriate by circuitry known in the art, which may be provided as part of the sensor or processing circuitry 26. In some examples, the signals may be converted to digital values and processed by processing circuitry 26 before being saved to memory 24 or uploaded to another device, such as via communication circuitry 30. In some cases, sensor 32 and/or sensors of sensor devices 16, 18, 20 generate a signal that is indicative of a physiological parameter measurement of patient 14 that varies as a function of patient activity. The relevant physiological parameters include, but are not limited to, heart rate, respiratory rate, electrocardiogram (ECG) morphology, respiration rate, respiratory volume, core temperature, a muscular activity level or subcutaneous temperature of the patient.

User interface 31 of computing device 12 may include one or more user input devices and one or more user output devices, e.g., a touch screen display or other display, a pointing device, a keyboard, or the like. Processing circuitry 26 may receive instructions, threshold values, or user-inputted symptom data via user interface 31. Processing circuitry 26 may also present a variety of information to a user, including an indication of whether patient 14 is likely experiencing a UTI, via user interface 31.

Although not illustrated in FIGS. 1 and 2, each of IMD 16, wearable sensor device 18, and/or sensor devices 20 may be configured similarly to computing device 12 in at least some aspects. For example, each of IMD 16, wearable sensor device 18, and/or sensor devices 20 may include processing circuitry, memory, a power source, communication circuitry, one or more sensors and, in some cases, a user interface, as described with respect to computing device 12. The techniques described herein as being performed by processing circuitry 26 of computing device may be performed by processing circuitry of any one or more devices described herein, alone or in any combination.

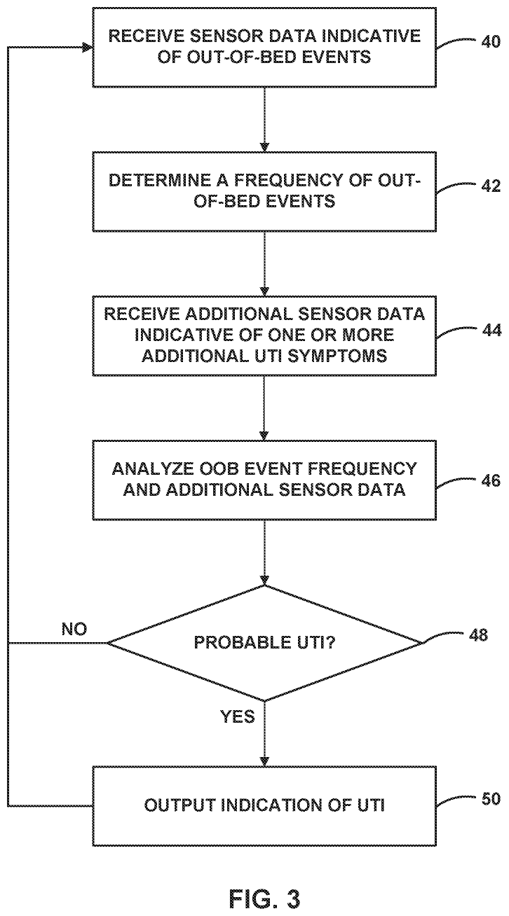

FIG. 3 is a flow diagram illustrating an example method for determining whether to provide an indication of the presence of a UTI in a patient, in accordance with techniques of this disclosure. Although described primarily as being performed by processing circuitry 26 of computing device 12 (FIGS. 1-2), some or all of the example method of FIG. 3 may be performed by processing circuitry of IMD 16, wearable sensor device 18, and/or sensor devices 20 (FIG. 1).

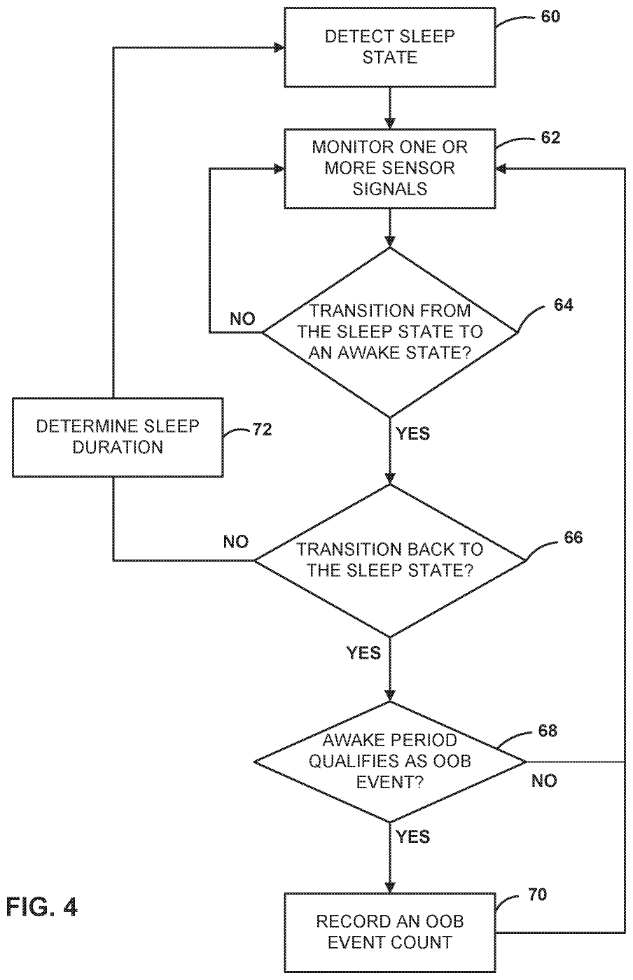

Processing circuitry 26 receives sensor data indicative of OOB events (40). For example, computing device 12 may receive sensor data from any number or type of sensor devices, such as an activity-level sensor, a body-position (posture) sensor, a heartrate, heartrate volume, or blood-pressure sensor, a thermometer, a motion sensor, a pressure sensor, a magnetometer or compass, an electrode, or a PPG sensor, as non-limiting examples. Based on the received sensor data, processing circuitry 26 may determine a number and/or frequency of OOB events for patient 14, indicating that patient 14 is awaking during a nighttime period to void their bladder, commonly known as “nocturia” (42). An example technique for determining a frequency of OOB events is described with respect to FIG. 4. In some examples, processing circuitry of a sensor device that includes activity sensors, e.g., IMD 16 and/or sensor device 18, may detect when patient 14 is within a sleep state, detect OOB events while patient 14 is within the sleep state and, in cases, determine frequencies of OOB events during the sleep states, e.g., on a per-sleep state or per-day basis.

In some examples, processing circuitry 26 may receive additional sensor data that is indicative of one or more additional UTI symptoms, such as data from any or all of the sensor devices previously discussed (44). For example, as discussed in greater detail above with respect to FIG. 2, processing circuitry 26 may analyze sensor data to identify additional UTI symptoms, such as, but not limited to, fatigue and/or tremors; fever and/or chills; agitation and/or restlessness; lower back pain; painful urination (e.g., a “burning” sensation); and/or presyncope and/or syncope (e.g., loss of consciousness and/or falls). Although included in the example of FIG. 3, in some examples, processing circuitry 26 does not receive additional sensor data and instead, for example, determines whether to provide an indication of UTI based on OOB frequency alone.

Processing circuitry 26 may analyze the OOB event frequency and, in some examples, the additional sensor data (46). In some examples, processing circuitry 26 may compare the OOB event frequency and additional UTI symptom data to one or more respective thresholds, as described above. Based on the analysis, e.g., based on whether the one or more thresholds, processing circuitry 26 determines whether UTI is probable in patient 14 (48). If processing circuitry 26 determines UTI is probable in patient 14 (YES of 48), processing circuitry 26 provides an indication of UTI to one or more users, such as the patient or a caregiver, e.g., via communication circuitry 30 and/or user interface 31 (50) For example, computing device 12 may output data to a display indicating the probability of a UTI, such that patient 14 may seek treatment as appropriate, or such that a physician may provide treatment as appropriate.

FIG. 4 is a flow diagram illustrating an example method for determining a frequency of OOB events by a patient according to techniques of this disclosure. FIG. 4 is primarily described with respect to computing device 12 (FIG. 2) and implantable sensor device, such as an implantable medical device (IMD) 16 (FIG. 1), however, it should be understood that the description similarly applies to any other external or implantable sensor devices that generate a signal indicative of a physiological patient parameter indicative of patient activity, as previously described.

Processing circuitry of IMD 16 detects a sleep state of patient 14 (60). A “sleep state” includes both a phase when patient 14 is attempting to sleep and when patient 14 is asleep. Because nocturia may disrupt the patient's rest by causing patient 14 to get up to urinate when patient 14 is attempting to sleep, counting the number of interruptions while patient 14 is attempting to sleep may be just as important in evaluating the severity of the nocturia as the number of interruptions when patient 14 is asleep.

Processing circuitry of IMD 16 may identify when patient 14 is attempting to sleep in a variety of ways. For example, the processing circuitry may identify the time that patient begins attempting to fall asleep based on an indication received from a patient 14. For example, patient 14 may provide an input via input keys or a display of computing device 12, a patient programmer device, cellular telephone, or another computing device, which may then be transmitted to IMD 16. In another example, patient 14 taps the general implant site of IMD 16 to indicate that the patient is attempting to sleep. As described in commonly-assigned U.S. patent application Ser. No. 11/755,587, entitled, “VOIDING EVENT IDENTIFICATION BASED ON PATIENT INPUT” to Martin T. Gerber, incorporated herein by reference in its entirety, tapping IMD 16 a certain number of times or in a certain pattern may cause processing circuitry 26 within IMD 16 to record the date and time of the tapping or activate a feature of IMD 16.

In other examples, the processing circuitry detects the sleep state (60) by identifying the time that a patient 14 begins attempting to fall asleep based on the activity level of patient 14 indicated via one or more sensors of IMD 16, such as one or more accelerometers and/or heart rate sensors. A relatively low level of activity indicates that patient 14 has likely entered a sleep state. The low level of activity may be cross-checked with the time of day (i.e., if IMD 16 includes a clock) or the posture of patient 14 in order to confirm that patient 14 is entering a sleep state and not merely inactive. Techniques for determining the posture of patient 14 is described in detail below.

One technique processing circuitry of IMD 16 may employ to detect a sleep state is to identify a time when the activity level of a patient 14 falls below a threshold activity level value stored in a memory and determine whether the activity level remains substantially below the threshold activity level value for a threshold amount of time stored in the memory. In other words, a patient 14 remaining inactive for a sufficient period of time may indicate that patient 14 is attempting to fall asleep, or has fallen asleep. If the processing circuitry determines that the threshold amount of time is exceeded, the processing circuitry may identify the time at which the activity level fell below the threshold activity level value as the time that the sleep state of patient 14 begins.

In some examples, the sensor(s) of IMD 16 may include one or more electrodes that generate an EMG signal as a function of muscle electrical activity, which may indicate the activity level of a patient. The electrodes may be, for example, located in the legs, abdomen, chest, back or buttocks of a patient 14 to detect muscle activity associated with walking, running or the like. The electrodes may be coupled to IMD 16 wirelessly or by a lead or, if IMD 16 is implanted in these locations, integrated with a housing of IMD 16. When the processing circuitry of IMD 16 determines that the muscle electrically activity falls below a threshold, the processing circuitry may determine that patient 14 has entered a sleep state.

Bonded piezoelectric crystals located in the legs, abdomen, chest, back or buttocks of a patient 14 generate signals as a function of muscle contraction in addition to body motion, footfalls or other impact events. Consequently, use of bonded piezoelectric crystals to detect activity of a patient 14 may be preferred in some examples in which it is desired to detect muscle activity in addition to body motion, footfalls or other impact events. Thus, in one example, the sensor(s) of IMD 16 include one or more bonded piezoelectric crystals that are coupled to IMD 16 wirelessly or via a lead, or piezoelectric crystals may be bonded to the housing of IMD 16 if IMD 16 is implanted in these areas, e.g., in the back, chest, buttocks or abdomen of patient 14. If IMD 16 is also used to deliver stimulation therapy to control the function of a bladder, however, IMD 16 may be implanted proximate to the bladder, rather than in the back, chest, buttocks or abdomen of patient 14.

In some examples, the processing circuitry of IMD 16 determines whether patient 14 is attempting to fall asleep based on the posture of patient 14, i.e., whether patient 14 is or is not recumbent. In such examples, the sensors of IMD 16 may include a plurality of accelerometers (e.g., one, two or three axis accelerometers), gyros, or magnetometers oriented orthogonally that generate signals which indicate the posture of a patient 14. In addition to being oriented orthogonally with respect to each other, each sensor that is used (if multiple sensors are used) to detect the posture of a patient 14 may be generally aligned with an axis of the body of the patient 14. In one example, the sensors of IMD 16 comprise three orthogonally oriented posture sensors.

When sensors include one or more accelerometers, for example, that are aligned in this manner, the processing circuitry of IMD 16 may monitor the magnitude and polarity of DC components of the signals generated by the accelerometers to determine the orientation of patient 14 relative to Earth's gravity, e.g., the posture of patient 14. In particular, the processing circuitry of IMD 16 may compare the DC components of the signals to respective threshold values stored in the memory of IMD 16 to determine whether patient 14 is or is not recumbent. Further information regarding use of orthogonally aligned accelerometers to determine patient posture may be found in a commonly assigned U.S. Pat. No. 5,593,431, which issued to Todd J. Sheldon, incorporated herein by reference in its entirety.

Other motion detection sensors that may generate a signal that indicates the posture of patient 14 include bonded piezoelectric crystals that generate a signal as a function of contraction of muscles, and electrodes that generate an electromyogram (EMG) signal. Such sensors may be implanted in the legs, buttocks, abdomen, or back of patient 14. The signals generated by such sensors when implanted in these locations may vary based on the posture of patient 14, e.g., may vary based on whether the patient is standing, sitting, or lying down.

Further, the posture of patient 14 may affect the thoracic impedance of the patient. Consequently, IMD 16 may include electrodes that generate a signal as a function of the thoracic impedance of patient 14. Processing circuitry of IMD 16 may detect the posture or posture changes of patient 14 based on the signal that is indicative of the thoracic impedance.

Additionally, changes of the posture of patient 14 may cause pressure changes with the cerebrospinal fluid of the patient. Consequently, IMD 16 may include pressure sensors coupled to one or more intrathecal or intracerebroventricular catheters, or pressure sensors coupled to IMD 16 wirelessly or via a lead. CSF pressure changes associated with posture changes may be particularly evident within the brain of the patient, e.g., may be particularly apparent in an intracranial pressure (ICP) waveform.

In some examples, processing circuitry of IMD 16 considers both the posture and the activity level of patient 14 when determining whether patient 14 is attempting to fall asleep, and thus, the beginning of the sleep state. For example, the processing circuitry may determine whether a patient 14 is sleeping based on a sufficiently long period of sub-threshold activity, as described above, and may identify the time that patient began attempting to fall asleep as the time when patient 14 became recumbent prior to the determination that the patient is sleeping. Any of a variety of combinations or variations of these techniques may be used to determine to detect the sleep state, and a specific one or more techniques may be selected based on the sleeping and activity habits of a particular patient.

When IMD 16 detects the sleep state (60), IMD 16 monitors signals received from one or more sensors (62) in order to detect whether the patient's sleep state is interrupted, and in particular, whether patient 14 gets up at any time during the sleep state to void. The sensors monitored to detect whether the sleep of patient 14 is interrupted may include any of the sensors previously described with respect to step (60), or may additionally or alternatively include other sensors, as described throughout this disclosure.

In one example, processing circuitry of IMD 16 monitors a sensor signal indicative of a patient motion, e.g., one or more accelerometer signals. In addition or instead of monitoring a signal from a patient motion sensor, the processing circuitry of IMD 16 may monitor a patient activity level by monitoring one or more physiological parameters of patient 14 that vary as a function of patient activity, such as heart rate, electrocardiogram (ECG) morphology, respiration rate, respiratory volume, core temperature, subcutaneous temperature or muscle activity.

Processing circuitry of IMD 16 may determine a patient activity level based on the signal from the sensor(s). In one example, the processing circuitry determines a patient activity level by sampling the signal and determining a number of activity counts during the sample period. In one example, the processing circuitry compares the signal to one or more amplitude thresholds stored within a memory. Processing circuitry of IMD 16 may identify each threshold crossing as an activity count. Where the processing circuitry compares the sample to multiple thresholds with varying amplitudes, the processing circuitry may identify crossing of higher amplitude thresholds as multiple activity counts. Using multiple thresholds to identify activity counts, the processing circuitry may be able to more accurately determine the extent of patient activity. In examples in which IMD 16 includes motion sensor in the form of a mercury switch, the processing circuitry of IMD 16 may identify the number of switch contacts indicated during the sample period as the number of activity counts. Processing circuitry of IMD 16 may store the determined number of activity counts in memory as an activity level in addition to or instead of storing the signals generated by the sensor(s).

In examples in which a sensor of IMD 16 generates a signal indicative of a physiological parameter of a patient that varies based on activity, processing circuitry of IMD 16 may monitor a signal from the sensor and determine a physiological parameter measurement based on the signal. The physiological parameter measurement may be mean or median values of the physiological parameter over a certain period of time. Based on the physiological parameter values, the processing circuitry may determine an activity level by comparing the determined physiological parameter measurement to one or more thresholds stored within memory. A first threshold may indicate a first activity level, a second a threshold may indicate a second activity level that is greater than the first activity level, and so forth for as many activity levels as desired. Processing circuitry of IMD 16 may compare the measured physiological parameter to the thresholds to determine the activity level corresponding to the measured physiological parameter. For example, if the measured parameter exceeds the second threshold, but not a third threshold, the measured parameter falls within the second activity level.

If one or more physiological parameters are measured to determine a patient activity level, the physiological parameter measurements that represent different activity levels may differ between patients, depending on the type of physiological parameter. For example, patients that are physically fit may have a different heart rate at an elevated activity state than patients that are not physically fit. Thus, between those two groups of patients, using the same heart rate threshold for both patients as an indicator of activity level may not be entirely accurate. Accordingly, it may be desirable to modify the thresholds to a particular patient.

In some examples, regardless of whether IMD 16 monitors patient motion or one or more physiological parameters, processing circuitry of IMD 16 may compare the signal generated by the sensor(s) to one or more amplitude thresholds, where each threshold crossing counts as an activity count, and the total number of activity counts indicates the patient activity level.

Based on the monitored patient activity level, the processing circuitry of IMD 16 determines whether patient 14 transitions from the sleep state to an awake state (64). The “awake state” is associated with an activity level that is higher than sedentary, in order to properly reflect when patient 14 physically walked or otherwise moved to another location (e.g., a bathroom) to void. As described in further detail below, in one example, IMD 16 determines whether patient 14 transitioned from the sleep state to the awake state (64) by comparing the patient activity level to an awake threshold.

If IMD 16 determines that patient 14 did not transition from a sleep state to an awake state, but, rather, patient 14 is still in the sleep state (NO of 64), IMD 16 may continue monitoring the activity level (62). If IMD 16 determines that patient 14 transitioned from a sleep state to an awake state, IMD 16 continues to monitor the activity level to determine whether patient 14 transitions back to a sleep state within a certain amount of time, e.g., indicative of a continuation of the current sleep duration rather than an end to the patient's attempt to sleep (66). Fluctuations between a sleep state and an awake state indicate that the patient's sleep state was interrupted. Limiting the amount of time in which patient 14 may return to the sleep state from the awake state helps to ensure that the sleep state did not end, but rather, the sleep state was interrupted.

As described in further detail below, in one example, IMD 16 determines whether patient 14 transitioned from an awake state to a sleep state (66) by comparing the patient activity level to an awake threshold. In another example, IMD 16 determines whether patient 14 transitioned from the awake state to the sleep state (66) by monitoring a sleep metric that indicates a probability that patient 14 is asleep or awake.

Examples of circuits and techniques that may be used to detect transitions between a sleep state and awake state based on various physiological parameters or other indicators of activity levels are described in U.S. Pat. No. 8,308,661, entitled “COLLECTING ACTIVITY AND SLEEP QUALITY INFORMATION VIA A MEDICAL DEVICE”, which is incorporated herein by reference in its entirety.

In some examples, IMD 16 may determine whether patient 14 transitioned between an awake state and a sleep state (64, 66) by comparing a determined patient activity level to an awake threshold. Processing circuitry of IMD 16 compares the patient activity level to an awake threshold (64), which is an activity level at or above which the patient is not only awake, but indicates the patient is active (versus sedentary). Because the relevant determination is whether the patient's sleep state is interrupted because of nocturia, the relevant awake threshold is an activity level that indicates patient 14 not only is awake, but is active, rather than sedentary. For example, the awake threshold is an activity level that indicates patient 14 is getting out of bed to void or walking to a bathroom. The activity level for patient 14 during the sleep state may differ from the activity levels of other patients, and accordingly, the awake threshold may be tailored to a particular patient. Alternatively, the awake threshold may be common to two or more patients.

If the patient activity level does not exceed the awake threshold (NO of 64), IMD 16 continues to monitor the activity level (62) until the patient activity level exceeds the awake threshold, if ever. After determining that patient 14 awoke from a sleep state, IMD 16 monitors the activity level to ensure that patient 14 returns to a sleep state (66) and that the sleep state did not end. In particular, if the patient activity level exceeds the awake threshold (YES of 64), IMD 16 continues to monitor the activity level to determine whether a subsequent patient activity level falls below the awake threshold within a certain amount of time (66). The amount of time may be determined by the clinician or other user, and may be, for example, less than twenty minutes, less than thirty minutes, less than one hour or less than two hours.

In an example in which IMD 16 includes one or more sensors that generates a signal indicative of patient motion, the activity thresholds may be associated with gross motor activity of the patient, e.g., walking, running or the like. Accordingly, it is highly unlikely that patient 14 is in a sleep state if the activity level exceeds the threshold associated with a motor activity such as walking. In some examples, one or more sensors of IMD 16 may be used to determine a posture of patient 14, with certain upright postures, or transitions to and from upright postures, indicating that the patient has awoken and returned to sleep.

In an example in which IMD 16 utilizes one or more physiological parameter sensors to monitor an activity level of patient 14, the detected values of physiological parameters of patient 14, such as heart rate, ECG morphological features, respiration rate, respiratory volume, blood pressure, blood oxygen saturation, partial pressure of oxygen within blood, partial pressure of oxygen within cerebrospinal fluid, muscular activity and tone, core temperature, subcutaneous temperature, arterial blood flow, brain electrical activity, eye motion, and galvanic skin response may discernibly change when patient 14 falls asleep or awakes. Some of these physiological parameters may be at low values when patient 14 is asleep. Further, the variability of at least some of these parameters, such as heart rate and respiration rate, may be at a low value when the patient is asleep.

Consequently, in order to detect when patient 14 falls asleep and wakes up, processing circuitry 26 may monitor one or more of these physiological parameters, or the variability of these physiological parameters, and detect the discernable changes in their values associated with a transition between a sleep state and an awake state. In some examples, processing circuitry 26 may determine a mean or median value for a parameter based on values of a signal over time, and determine whether patient 14 is asleep or awake based on the mean or median value. Processing circuitry 26 may compare one or more parameter values or parameter variability values to thresholds stored in memory 24 to detect when patient 14 transitions from the sleep state to the awake state or vice versa (64, 66). The thresholds may be absolute values of a physiological parameter, or time rate of change values for the physiological parameter, e.g., to detect sudden changes in the value of a parameter or parameter variability. In some examples, a threshold used by processing circuitry 26 to determine whether patient 14 is asleep may include a time component. For example, a threshold may require that a physiological parameter be above or below a threshold value for a period of time before processing circuitry 26 determines that patient is awake or asleep.

As an alternative to comparing activity levels to thresholds, the relative changes in a patient's activity level may be used to determine when the patient is in an elevated activity state that reflects an interruption in the sleep state. For example, after determining a sleep state has commenced, processing circuitry 26 may monitor the activity levels for changes in the activity level, where an increase or sudden change in one or more of heart rate, heart rate variability, respiration rate, respiration rate variability, blood pressure, ECG morphological features or muscular activity indicates an increase in activity associated with a transition from the sleep state to the awake state, or vice versa. The rate or amount of change in the physiological parameter or variability may be compared to a threshold stored in memory 24.

In another example, processing circuitry 26 of IMD 16 determines whether patient 14 transitioned from the awake state to the sleep state (66) by monitoring a plurality of physiological parameters, and determining a value of a metric that indicates the probability that the patient 14 is asleep for each of the parameters based on a value of the physiological parameter. Because the physiological parameter varies as a function of patient activity, the metric also varies as a function of patient activity, and therefore, is one way of monitoring the patient activity level.

In another example, processing circuitry 26 of IMD 16 determines a number of interruptions in a sleep state of patient 14 by analyzing one or more sleep metrics that indicate the probability that the patient 14 is asleep. In particular, processing circuitry 26 determines a sleep metric by applying a function or look-up table to the current, mean or median value, and/or the variability of each of a plurality of physiological parameters determined based on signals from sensor(s) 32. A sleep metric value may be a numeric value, and in some examples may be a probability value, e.g., a number within the range from 0 to 1, or a percentage value.

Processing circuitry 26 may average or otherwise combine the plurality of sleep metric values to provide an overall sleep metric value. In some examples, processing circuitry 26 may apply a weighting factor to one or more of the sleep metric values prior to combination. Use of sleep metric values to determine when a patient is asleep based on a plurality of monitored physiological parameters is described in greater detail in the commonly-assigned U.S. patent application Ser. No. 11/691,405, entitled “DETECTING SLEEP,” and filed on Mar. 26, 2007, which is incorporated herein by reference in its entirety.

Based on a determined sleep metric, processing circuitry 26 may detect an initial sleep state (60), such as by comparing an overall sleep metric value or a particular sleep metric value to one or more threshold values stored in memory 24 to determine when patient 14 has entered a sleep state. Processing circuitry 26 may continue monitoring the sleep metric to determine whether the metric indicates the patient is awake and active (64). Again, the relevant determination is whether the patient's sleep was interrupted by an act of getting out of bed to void. Processing circuitry 26 may compare the overall sleep metric value to one or more threshold values stored in memory 24 to determine when patient 14 is asleep or awake and active. If not, processing circuitry 26 continues to monitor the one or more physiological parameters via sensor(s) 32 and determine sleep metric based on the sensed physiological parameters. If the sleep metric indicates the patient is awake, processing circuitry 26 determines whether a subsequent metric based on subsequently sensed physiological parameters measured within a certain amount of time from the time it is determined that patient 14 is awake indicate the patient is asleep (66). As previously described, processing circuitry 26 determines whether patient 14 returned to the sleep state within a certain amount of time in order to help ensure that any further awake counts are associated with the correct sleep event. If patient 14 returns to the sleep state, processing circuitry 26 records an awake count in memory 24. If not, processing circuitry 26 detects another sleep event by detecting another sleep state (60), and begins the process over for the other sleep event.

In another example of the process shown in FIG. 4, processing circuitry 26 of IMD 16 determines whether patient 14 transitioned from the awake state to the sleep state (66) by monitoring a posture of patient 14, where posture is one type of patient activity. After detecting the start of a sleep state (60), processing circuitry 26 may monitor the posture of patient 14. The posture of patient 14 may be determined via any of the techniques described above. For example, sensor 32 may comprise a multi-axis accelerometer that generates a signal indicative of the posture of patient 14.

Typically, patient 14 is recumbent when sleeping. Accordingly, a deviation from the recumbent posture may indicate that patient 14 is in an awake state and active, which indicates that patient 14 awoke to void. Thus, the process shown in FIG. 4 includes determining whether patient 14 is recumbent (60), and thus, asleep. However, if patient 14 sleeps in a posture other than recumbent, the other posture may be used as the baseline to detect a change in posture that indicates patient 14 is in an awake state. The posture data may be used in addition to a physiological parameter, such as muscle activity, to determine whether the patient's posture not only changed, but whether patient 14 got up to void. Examples in which processing circuitry 26 monitors both the posture and activity level of patient 14 may provide a good indication of circumstances surrounding a patient activity level. That is, monitoring both posture and activity data may provide a robust technique for determining whether patient 14 is in a sleep state or an awake state. In some cases, patient activity level alone may not clearly indicate whether patient 14 is transitioning between an awake and sleep state because patient 14 may be moving around while trying to fall asleep. Combining the activity level with a patient posture may indicate whether the patient is in the sleep state or awake state. For example, a relatively high level of activity (e.g., crossing a threshold indicating that patient 14 is “active”) while patient 14 is recumbent may indicate patient 14 is in the sleep state, but is having trouble falling asleep. On the other hand, a relatively high level activity while patient 14 is standing or otherwise upright may indicate patient 14 is in an awake state for the purposes of recording an awake count. As another example, a relatively low level of activity may indicate patient 14 is asleep, and detecting a recumbent posture may validate the sleep state determination.

If patient 14 is recumbent, and thus still in the sleep state, processing circuitry 26 may continue monitoring patient posture (62) until a change in posture is detected, if at all. If processing circuitry 26 determines that patient 14 is no longer recumbent, and, thus, patient 14 is awake, processing circuitry 26 may continue monitoring the posture to detect a change in posture back to the recumbent posture (66). If processing circuitry 26 detects a subsequent recumbent posture within a certain amount of time, such as less than one or two hours, processing circuitry 26 records an awake count in memory 24 of IMD 16.

Regardless of the specific technique by which IMD 16 determines a transition from a sleep state to an awake state (64) and back (66), in some examples, IMD 16 may additionally determine whether each “awake count” actually qualifies as an out-of-bed (OOB) event (68). For example, IMD 16 may further analyze the activity levels, other physiological parameters, and/or additional sensor data during each awake (e.g., active) period to determine whether the patient is actually out-of-bed due to a bladder voiding event, or for an unrelated reason. For example, IMD 16 may analyze a gross activity level, a number of steps indicated by a pedometer, or other activity pattern during an awake period that is relatively highly indicative of bathroom use. In other examples, IMD 16 may analyze additional sensor data, such as from a motion sensor within a bathroom, or a pressure sensor under a toilet seat, in order to determine whether a particular awake period qualifies as an out-of-bed bladder-voiding event (68).

If IMD 16 determines that the patient 14 transitioned back to a sleep state from an awake state (YES of 66), and in some examples, additionally determines that the transition was due to a bladder voiding event (YES of 68), processing circuitry 26 of IMD 16 records an OOB event in memory 24 (70). The amount of time within which patient 14 may transition back to the sleep state before IMD 16 records an OOB event (70) may be predetermined by a clinician, and may be specific to a particular patient, or may be used for more than one patient. For example, the amount of time to transition back to the sleep state prior to recording the OOB event (70) may be less than an hour or less than two hours. In general, the amount of time should be short enough to distinguish between the time between sleep events (e.g., the time between two separate nights of sleep) and the time it might take patient 14 to void and return to the sleep state. Limiting the amount of time in which patient 14 may fall back into the sleep state prior to recording an awake count helps ensure that the OOB events are associated with a single sleep event. The number of OOB events recorded in memory 24 and associated with a sleep event (e.g., one night of sleep versus multiple nights of sleep) generally represents the number of interruptions in the sleep state of patient 14 that are attributable to nocturia. Therefore, in one example, processing circuitry 26 determines a number of nocturia events based on the determined number of interruptions in the sleep state of patient 14. IMD 16 may determine whether patient 14 has contracted a UTI based on the number and/or frequency of recorded OOB events. For example, a higher number and/or frequency of OOB events may correspond to a higher probability of a UTI.

In addition to recording an OOB event (70), in some examples, processing circuitry 26 may record the date and time of the OOB event was generated. For example, IMD 16 may include a clock that is coupled to processing circuitry 26, which may obtain a clock signal from the clock to associate a timestamp with the OOB event. Processing circuitry 26 may associate a timestamp with a detected voiding event by sending a request signal to the clock. In response to receiving the control signal, the clock may generate a signal that represents the time. Alternatively, the clock may output the signal to processing circuitry 26 substantially continuously and processing circuitry 26 can examine the signal in response to recording an awake count. The clock may also be used to activate when processor 26 beings detecting a sleep state (60) and monitoring the patient's activity level (62), which may decrease the processor's power consumption.

In some cases, after an OOB event is recorded (70), thus indicating that a voiding event occurred, it may be unlikely that another voiding event will occur for a certain amount of time, such as thirty minutes to an hour, but the time may be more or less. Accordingly, in some examples, after recording an OOB event (70), processing circuitry 26 may enter into a “blanking” mode, which processing circuitry 26 waits a certain prescribed amount of time before detecting another sleep state and beginning the process over to detect a nocturia event. The prescribed amount of time may be determined by a clinician or a manufacturer of IMD 16. The blanking mode may help processing circuitry 26 conserve energy, which may help extend the useful life of IMD 16. On the other hand, a blanking state may not be useful for all patients.

If patient 14 did not transition back to the sleep state (NO of 66), for example, after a threshold period of time, processing circuitry 26 may identify the end of that particular sleep event (e.g., that nighttime period), and accordingly may determine a total sleep duration for that sleep event (72). For example, processing circuitry 26 may use the total sleep duration to determine a relative duration or frequency of OOB events, such as OOB events per hour, OOB events per sleep event (e.g., OOB events per night), or a total percent of each sleep duration event that is spent during OOB events. Processing circuitry 26 may then begin detecting a new sleep event (for example, during a new nighttime period) by detecting another sleep state (60), and begins the process over for the new sleep event. The process shown in FIG. 4 may be repeated for each sleep state detected by processing circuitry 26 of IMD 16, where each sleep state is associated with a new sleep event.

In some examples, IMD 16 does not determine whether patient 14 transitioned between a sleep state and awake state (64, 66) or record the OOB events (70), but instead provides monitored activity levels to a computing device, such as a clinician programmer or patient programmer. In such examples, the computing device may analyze activity stored within memory 24 of IMD 16 and determine the number of times patient 14 transitioned from the sleep state to the awake state (64) and back to the sleep state (66). In this way, the computing device may record awake counts and determine the number of interruptions in the sleep state of the patient. Computing device 12 (FIG. 2) may use the number of interruptions in the sleep state of the patient to determine the probability that patient 14 has contracted a UTI. Additionally, IMD 16 need not monitor the activity levels, but may instead store samples of the signals generated by sensor(s) 32. In such examples, the computing device may determine both activity levels and the number of awake counts for each sleep state.

In some examples in accordance with this disclosure, IMD 16 may determine whether patient 14 has contracted a UTI based on the number and/or frequency of recorded OOB events (70). For example, a higher number and/or frequency of OOB events may correspond to a higher probability of a UTI.

Various examples of the invention have been described. These and other examples are within the scope of the following claims.

What is claimed is:

1. A method comprising:
detecting out-of-bed events of a patient based on sensor data from at least one sensor device;
determining a frequency of the detected out-of-bed events for the patient; and
determining, based on the frequency of out-of-bed events for the patient, to provide a urinary tract infection (UTI) indication for the patient.

2. The method of claim 1, wherein the sensor data comprises activity-level data of the patient.

3. The method of claim 2, wherein detecting out-of-bed events for the patient comprises confirming that the out-of-bed event is associated with a bladder-voiding event by at least detecting:
a first increased activity level indicative of a first movement between a bedroom and a toilet;
a decreased activity level indicative of a bladder-voiding event; and
a second increased level indicative of a second movement between the toilet and the bedroom.

4. The method of claim 2, wherein the activity-level data comprises a heartrate of the patient.

5. The method of claim 1, wherein the at least one sensor device comprises a wearable sensor device.

6. The method of claim 1, wherein the at least one sensor device comprises an implantable medical device.

7. The method of claim 1, wherein the at least one sensor device comprises an accelerometer.

8. The method of claim 1, wherein the sensor data indicates a direction of motion of the patient, and wherein detecting at least one of the out-of-bed events comprises detecting:
a first direction of motion from a bedroom to a toilet; and
a second direction of motion from the toilet to the bedroom.

9. The method of claim 3, wherein detecting out-of-bed events for the patient comprises:
confirming that the out-of-bed event is associated with a bladder-voiding event by detecting a parameter comprising:
a time duration of the first increased activity level;
a cumulative activity amount of the first increased activity level; or
a number of steps taken by the patient during the first increased activity level; and
determining whether the parameter falls within a threshold window indicative of a bladder-voiding event.

10. The method of claim 1, wherein the sensor data comprises first sensor data, the method further comprising receiving, from the at least one sensor device, second sensor data indicative of a non-nocturia UTI symptom for the patient, wherein determining to provide the UTI indication comprises determining to provide the UTI indication based on the second sensor data.

11. The method of claim 10, wherein the non-nocturia UTI symptom comprises:
fatigue or tremors;
fever or chills;
agitation or restlessness;
lower back pain;
painful urination; or
presyncope or syncope.

12. A system comprising:
at least one sensor device configured to collect sensor data; and
processing circuitry configured to:
detect out-of-bed events of a patient based on the sensor data;
determine a frequency of the detected out-of-bed events for the patient; and
determine, based on the frequency of out-of-bed events for the patient, to provide a urinary tract infection (UTI) indication for the patient.

13. The system of claim 12, wherein the sensor data comprises activity-level data of the patient.

14. The system of claim 13, wherein detecting out-of-bed events for the patient comprises confirming that the out-of-bed event is associated with a bladder-voiding event by at least detecting:
a first increased activity level indicative of a first movement between a bedroom and a toilet;
a decreased activity level indicative of a bladder-voiding event; and
a second increased level indicative of a second movement between the toilet and the bedroom.

15. The system of claim 13, wherein the activity-level data comprises a heartrate of the patient.

16. The system of claim 12, wherein the at least one sensor device comprises a wearable sensor device.

17. The system of claim 12, wherein the at least one sensor device comprises an implantable medical device.

18. The system of claim 12, wherein the at least one sensor device comprises an accelerometer.

19. The system of claim 12, wherein the sensor data indicates a direction of motion of the patient, and wherein detecting at least one of the out-of-bed events comprises detecting:
a first direction of motion from a bedroom to a toilet; and
a second direction of motion from the toilet to the bedroom.

20. The system of claim 12, wherein detecting out-of-bed events for the patient comprises:
confirming that the out-of-bed event is associated with a bladder-voiding event by detecting a parameter comprising:
a time duration of the first increased activity level;
a cumulative activity amount of the first increased activity level; or
a number of steps taken by the patient during the first increased activity level; and
determining whether the parameter falls within a threshold window indicative of a bladder-voiding event.

21. The system of claim 12, wherein the sensor data comprises first sensor data, the processing circuitry further configured to receive, from the at least one sensor device, second sensor data indicative of a non-nocturia UTI symptom for the patient, wherein determining to provide the UTI indication comprises determining to provide the UTI indication based on the second sensor data.

22. The system of claim 21, wherein the non-nocturia UTI symptom comprises:
fatigue or tremors;
fever or chills;
agitation or restlessness;
lower back pain;
painful urination; or
presyncope or syncope.
